(19) United States
(12) Patent Application Publication
(10) Pub. No.: US 2023/0233091 A1
(43) Pub. Date: Jul. 27, 2023

(54) Systems and Methods for Measuring Vital Signs Using Multimodal Health Sensing Platforms

(71) Applicant: The Regents of the University of California, Oakland, CA (US)

(72) Inventors: Achuta Kadambi, Los Angeles, CA (US); Laleh Jalilian, Santa Monica, CA (US); Pradyumna Chari, Los Angeles, CA (US); Chinmay Talegaonkar, Los Angeles, CA (US); Doruk Karinca, Los Angeles, CA (US); Maxime Cannesson, Los Angeles, CA (US); Krish Kabra, Los Angeles, CA (US); Omid Salehi-Abari, Oakland, CA (US); Ashley Kita, Oakland, CA (US); Adnan Armouti, Los Angeles, CA (US)

(73) Assignee: The Regents of the University of California, Oakland, CA (US)

(21) Appl. No.: 18/002,097

(22) PCT Filed: Jun. 16, 2021
(86) PCT No.: PCT/US2021/037682

§ 371 Date: Dec. 16, 2022

Related U.S. Application Data
(60) Provisional application No. 63/177,229, filed on Apr. 20, 2021.
(60) Provisional application No. 63/039,887, filed on Jun. 16, 2020.

(51) Int. Cl. A61B 5/0205; A61B 5/00; G06T 7/246; G06T 7/00

(52) U.S. Cl. CPC A61B 5/02055; A61B 5/7267; A61B 5/7278; A61B 5/7221; G06T 7/246; G06T 7/0012; A61B 5/0816

(57) ABSTRACT

Systems and methods for measuring vitals in accordance with embodiments of the invention are illustrated. One embodiment includes a method for measuring vital signs. The method includes steps for identifying regions of interest (ROIs) from video data of an individual, generating temporal waveforms from the ROIs, analyzing the generated temporal waveforms to extract vital sign measurements, and generating outputs based on the analyzed temporal waveforms.

### CROSS REFERENCE TO RELATED APPLICATIONS

This application claims priority to U.S. Provisional Application Ser. No. 63/039,887, entitled “Methods and Apparatus for Extracting Vital Signs Using Smartphone Cameras” to Kadambi et al., filed Jun. 16, 2020 and to U.S. Provisional Application Ser. No. 63/177,229, entitled “MMHEALTH: Multimodal Health Sensing Platform” to Kadambi et al., filed Apr. 20, 2021; the disclosures of which are herein incorporated by reference in their entirety.

### FIELD OF THE INVENTION

The present invention generally relates to a system for measuring vital signs and, more specifically, a contactless system for measuring vital signs in a non-invasive manner using imaging and/or one or more different sensing modalities.

### BACKGROUND

The COVID-19 pandemic has accelerated the transition of healthcare delivery to a new model of remotely delivered care that embraces the benefits of digital and data technologies. Prior to the pandemic, clinical workflows and economic incentives supported and reinforced a face-to-face model of care, resulting in the congregation of patients in emergency departments and clinic waiting areas. This care structure contributes to spread of coronavirus to uninfected patients who are seeking evaluation and exposure of healthcare workers to infected patients. Contagious diseases like COVID-19 pose a serious threat to healthcare workers and all levels of support staff who come into contact with patients.

Patients with COVID-19 present with many non-specific symptoms including cough, fever, myalgias, and headache, but the clinical symptoms of tachycardia and tachypnea are concerning and warrant evaluation with pulse oximetry. Tachypnea may be a manifestation of hypoxia, and thus care teams that triage patients with COVID-19 will want to know pulse oximetry levels in their assessment, allowing health systems only to admit patients to the hospital for oxygen therapy if they demonstrate hypoxia. For all fields of medicine, digital tools can play crucial role in facilitating the monitoring of high risk patients and providing high-quality remote care.

However, as health care systems are transitioning to telemedicine and remotely delivered care, they lack the ability to obtain quantitative physiological vital sign data remotely. Vital sign data provides clinicians with valuable physiological data that is used to diagnose and treat various medical conditions. Key vital signs can include (but are not limited to) pulse oxygenation, heart rate, and respiration rate. “Heart Rate” is the speed of the heartrate in beats per minute. “Oxygenation” is the percentage of blood that is loaded with oxygen. More precisely oxygenation is the ratio of oxygenated hemoglobin to total hemoglobin, reported in units of oxygen saturation (SpO2). “Respiration Rate” is the number of breaths an individual takes per minute.

### SUMMARY OF THE INVENTION

Systems and methods for measuring vital signs in accordance with various embodiments of the invention are illustrated. One embodiment includes a method for measuring vital signs, including steps for identifying regions of interest (ROIs) from video data of an individual, generating temporal waveforms from the ROIs, analyzing the generated temporal waveforms to extract vital sign measurements, and generating outputs based on the analyzed temporal waveforms.

In a further embodiment, the method also includes steps for capturing the video, wherein capturing the video comprises capturing video of an individual, analyzing the captured video to determine whether a quality exceeds a given threshold, and when the quality does not exceed the given threshold, providing instructions to recapture the video.

In still another embodiment, the method further includes steps for processing the captured video, wherein processing the captured video includes at least one of illumination normalization and motion stabilization.

In a still further embodiment, the method further includes steps for generating a motion cue video and a color cue video, wherein processing the color cue video includes motion stabilization and processing the motion cue video does not include motion stabilization.

In yet another embodiment, identifying ROIs from video data includes performing segmentation using a convolutional neural network (CNN).

In a yet further embodiment, generating temporal waveforms from the ROIs comprises tracking motion of facial features within the ROIs between frames of video, and calculating velocity vectors based on the tracked motion.

In another additional embodiment, analyzing the generated temporal waveforms to extract vital sign measurements includes performing component analysis and frequency filtering to identify patterns in the video.

In a further additional embodiment, the identified patterns include at least one of changes in at least one channel of the video data and periodic motion in the head and neck region.

In another embodiment again, generating outputs based on the analyzed temporal waveforms includes providing a notification when at least one of the vital sign measurements exceeds a given threshold.

One embodiments includes a multi-modal system for diagnosing disease, including: several different types of sensors, including: an RGB camera that captures image data; a near infrared imaging (NIR) camera that captures NIR image data; a thermal imaging (TI) camera that captures thermal image data; at least one processor; memory storing a disease diagnosis application, wherein the disease diagnosis application directs the processor to: perform an r-PPG process that generates a final r-PPG signal by: generating a first r-PPG signal estimate using a first process that includes using a multi-modal transformer model, wherein the multimodal transformer is used to train on the image data, the NIR image data, and the thermal image data to obtain the r-PPG estimate, generating a second r-PPG signal estimate using a second different process that includes estimating the second r-PPPG from RGB data, NIR data, and TI data separately and aggregating them together, determining the final r-PPG signal estimate based on the first r-PPG estimate and the second r-PPG signal estimate; perform a respiratory rate process that generates a final respiratory waveform signal by: generating a first respiratory waveform estimate using a multimodal transformer, wherein the multimodal transformer is trained on the NIR image data and the TI image data; generating a second respiratory waveform estimate from the NIR image data and the TI image data separately and then aggregating them together; and determine the final respiratory waveform signal based on the first respiratory waveform estimate and the second respiratory waveform estimate; perform a blood oxygenation process that generates an oxygen saturation estimation; perform a decision level fusion process that includes a plurality of competing aggregator multimodal transformer models including a first model and a second model, wherein the first model receives as inputs the outputs from the r-PPG process, the respiratory rate process, and the blood oxygenation process; wherein the second model receives as inputs raw data from the plurality of different types of sensors.

In a further embodiment, the blood oxygenation pipeline computes a band limited amplified skin reflectance variations at a 3D region of interest and adds to original grey value variations.

In still a further embodiment, the system includes an RF sensor that captures RF data; and an audio sensor that captures audio data; wherein the disease diagnosis application direct the processor to perform an acoustic features process that: divides audio data captured by the audio sensor into a first section with continuous speech and a second section with forced coughs; train a first audio model using the first section; and train a second audio model using the second section.

In still a further embodiment, a Poisson mask is applied to the audio data, wherein the Poisson mask equation is:

$$M ( {I}_{x} ) = P o i s s ( λ ) {I}_{x} \qquad ( l )$$

$$Poiss ( X = k ) = \frac{{λ}^{k} {exp}^{- k}}{k !}$$

wherein the Poisson Mask applied to a specific MFCC value Ix can be calculated by multiplying this value by a random Poisson distribution of parameters Ix and λ, where λ is the average value of the entire MFCC set.

In still a further embodiment, the system includes a temperature pipeline.

In still a further embodiment, the decision level fusion process applies fuzzy aggregation to fuze different types of data and wherein a discrete Choquet Integral (CI) is used to fuze the classifier inputs and the highest confident class is selected, wherein the discrete Choquet Integral is:

C_{g}^{j}(d)=Σₜ₌₁^{T}d_{((t),j)}(x)[g(Aₜ)−g(Aₜ₋₁)],

where C_{g}^{j}(d) is the integral for class j and fuzzy measure g, the inputs are sorted in decreasing order, and Aₜ is the set of inputs from (1) to (t).

In a further embodiment, the decision level fusion process applies a Linear Order Static Neuron (LOSN) process.

One embodiment includes a multi-modal system for diagnosing disease, including several different types of sensors that capture different types of data, including a first type of data and a second type of data; generate a first vital sign estimate using a first process that includes using a multi-modal transformer model, wherein the multimodal transformer is used to train on the first type of data and the second type of data; generate a second vital sign estimate using a second different process that includes estimating the second vital sign estimate using the first type of data and the second type of data separately and then aggregating them together; perform decision level fusion; generate a disease diagnosis.

In a further embodiment, the first process includes generating a first r-PPG signal estimate that includes using a multi-modal transformer model, wherein the multimodal transformer is used to train on image data, NIR image data, and thermal image data to obtain the first r-PPG estimate; where the second process includes generating a second r-PPG signal estimate that includes estimating a second r-PPPG from RGB data, NIR data, and TI data separately and aggregating them together.

In a further embodiment, the multi-modal system further includes performing a respiratory rate process that generates a final respiratory waveform signal by: generating a first respiratory waveform estimate using a multimodal transformer, wherein the multimodal transformer is trained on NIR image data and the TI image data; generating a second respiratory waveform estimate from the NIR image data and the TI image data separately and then aggregating them together; and determine the final respiratory waveform signal based on the first respiratory waveform estimate and the second respiratory waveform estimate.

In a further embodiment, the multi-modal system further includes performing a blood oxygenation process that generates an oxygen saturation estimation; perform a decision level fusion process that includes a plurality of competing aggregator multimodal transformer models including a first model and a second model, wherein the first model receives as inputs the outputs from the r-PPG process, the respiratory rate process, and the blood oxygenation process; wherein the second model receives as inputs raw data from the plurality of different types of sensors.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates an example of a vital sign measurement system that measures vital signs of one or more patients in accordance with an embodiment of the invention.

FIG. 2 illustrates an example use case of a vital sign measurement system in accordance with an embodiment of the invention.

FIG. 3 illustrates an example of a vital sign measurement device that can measure vital signs of a patient in accordance with an embodiment of the invention.

FIG. 4 illustrates an example of a vital sign measurement application that measures vitals in accordance with an embodiment of the invention.

FIG. 5 conceptually illustrates a process for measuring vitals in accordance with an embodiment of the invention.

FIG. 6 conceptually illustrates an overview of a process for data collection and disease diagnosis in accordance with an embodiment of the invention.

FIG. 7 conceptually illustrates a COVID-19 diagnosis multimodal pipeline in accordance with an embodiment of the invention.

FIG. 8 conceptually illustrates a remote photoplethysmography (r-PPG) pipeline architecture, where images are RGB, NIR, and TI data in accordance with an embodiment of the invention.

FIG. 9 conceptually illustrates a respiratory rate pipeline architecture, where images are NIR and TI data in accordance with an embodiment of the invention.

FIG. 10 conceptually illustrates an acoustic features pipeline architecture in accordance with an embodiment of the invention.

FIG. 11 conceptually illustrates a multi-modal health (MMHealth) device mounted on an optical slab in accordance with an embodiment of the invention.

FIG. 12 conceptually illustrates a multi-modal IoT network architecture for cloud computing in accordance with an embodiment of the invention.

FIG. 13 conceptually illustrates separate, controlled environments used for data collection in accordance with an embodiment of the invention.

FIG. 14 conceptually illustrates a process for disease diagnosis using a multi-modal pipeline in accordance with an embodiment of the invention.

FIG. 15 conceptually illustrates a heart rate estimation process that includes a combination technique in the pipeline that incorporates skin diffuse information weighting, in addition to SNR weighting in RGB space, to achieve robust r-PPG performance across skin tones in accordance with an embodiment of the invention.

### DETAILED DESCRIPTION

Turning now to the drawings, systems and methods for measuring vital signs of an individual are disclosed herein. Methods in accordance with a variety of embodiments of the invention can measure various vital signs, such as (but not limited to) heart rate, pulse oxygenation, respiration rate, and/or blood pressure. In many embodiments, systems and methods can provide a unified, image-based technology to track and measure vital signs.

In a number of embodiments, a contactless camera-based vital sign detection approach is utilized that relies upon two distinct types of body cues: (i) subtle motion cues, and (ii) subtle color variation cues. Periodic facial motion cues can be caused by ballistic forces associated with blood flow through the carotid artery, whereas motion cues in the chest and shoulders can be the direct consequence of lung expansion and contraction. In numerous embodiments, these subtle motion cues can be captured by a camera and utilized to estimate heartbeat and respiratory rate respectively. Color variation cues can be caused by variations in blood light absorption due to changes in either blood volume or blood oxygenation. Hence, in many embodiments, these color cues can be utilized to estimate blood oxygenation levels and blood pressure.

Systems and methods in accordance with a number of embodiments of the invention can simultaneously detect key vital signs with a process that rejects common nuisance factors. Nuisance factors (e.g., illumination variation) are a nuisance to the robust estimation of multiple vital signs. In many embodiments, processes can use a given vital measurement to measure a nuisance factor, which can be used to measure other vital sign measurements. For example, since heart rate can be the easiest to measure, the illumination nuisance can be estimated after obtaining the correct heart rate. This information can then be passed on to more difficult vital signs since the illumination will have similar nuisance effects on the estimation of these other variables.

In a number of embodiments, systems and methods are provided for “human in the loop” image processing. Systems in accordance with some embodiments of the invention can be designed to guide a user to capture video data of a region where vitals can be tracked. In various embodiments, this can be done by identifying a nuisance factor related to illumination (e.g., poor lightning in the room) and scoring the perceived accuracy upon user change. For example, the user/patient could be prompted to turn on additional lighting in their environment.

Systems and methods in accordance with various embodiments of the invention can provide processes for motion stabilization. Since key vital signs, like heart rate and respiration rate can rely on motion cues, nuisance factors such as camera shake, background motion, and voluntary patient motion can affect the estimation of these vital signs. In contrast to previous work, processes in accordance with many embodiments of the invention can remove camera shake and background motion. In certain embodiments, using real-time optical flow fields, processes in accordance with many embodiments of the invention can align video frames with pixel-level accuracy (or sub-pixel level accuracy), while exploiting prior knowledge in human facial shape (e.g., a mixture of flat regions and rough contours) to more reliably track features from optical flow.

In several embodiments, novel methods are provided for region of interest (ROI) selection. It can be important to find the right region on each individual for which to extract vital signs. Previous work considers the problem of ROI selection (e.g. face detection) and signal extraction tasks independently. When these two steps are combined, systems and methods in accordance with many embodiments of the invention can achieve increased robustness to illumination variance and subject skin tones. Systems and methods in accordance with various embodiments of the invention can perform an adaptive weighted sampling on different regions and features of the face. In certain embodiments, weighting can be optimized using reliability metrics designed using various factors, such as (but not limited to) motion variance, illumination statistics, skin-tone complexion, and/or output vital sign accuracy.

Systems and methods in accordance with a variety of embodiments of the invention can provide novel processes for signal extraction. For periodic vitals, like heart rate and respiratory rate, after reliable facial region and feature extraction, processes in accordance with some embodiments of the invention can select an appropriate periodic signal among a host of candidates obtained through a signal processing pipeline. Processes in accordance with certain embodiments of the invention can use a combination of statistical priors and machine learning to accurately identify the heartbeat signal. In many embodiments, processes can analyze a frequency spectrum distribution of candidate signals and choose the signal that best resembles a heartbeat in the frequency domain. In a variety of embodiments, processes can identify a neural network embedding space in which the heartbeat signal has maximum distance separation from nuisance signals.

Many embodiments provide for a multimodal health (MMHealth) sensor platform for remote diagnosis of various medical conditions. In many embodiments, the MMHealth platform can include (but are not limited to) 3D and/or 2D image sensors, at various spectral bandwidths, along with mixed modalities such as radio frequency and acoustic chipsets. Accordingly, many embodiments of the multimodal health platform provide an end-to-end system that includes a multimodal sensor stack with networking capabilities for remote disease diagnosis. In several embodiments, the system may use multiple modalities for estimating different vital sign measurements such as individual heart rates, where the addition of the multiple modalities improves classification performance. In a number of embodiments, the multimodal data can be used to generate robust remote photoplethysmography (r-PPG) signals, respiratory rate and blood oxygenation features. These three features, along with the unimodal and data-mined features, can then be used to train multimodal transformer models and generate a final infectious disease diagnosis.

Systems and methods in accordance with several embodiments of the invention can provide technical solutions to privacy and signal detection in the technical field of image-based vital sign measurement. In certain embodiments, systems and methods can provide unconventional and novel methods for measuring vital signs through a contactless imaging system.

### Systems for Measuring Signs

### Vital Sign Measurement System

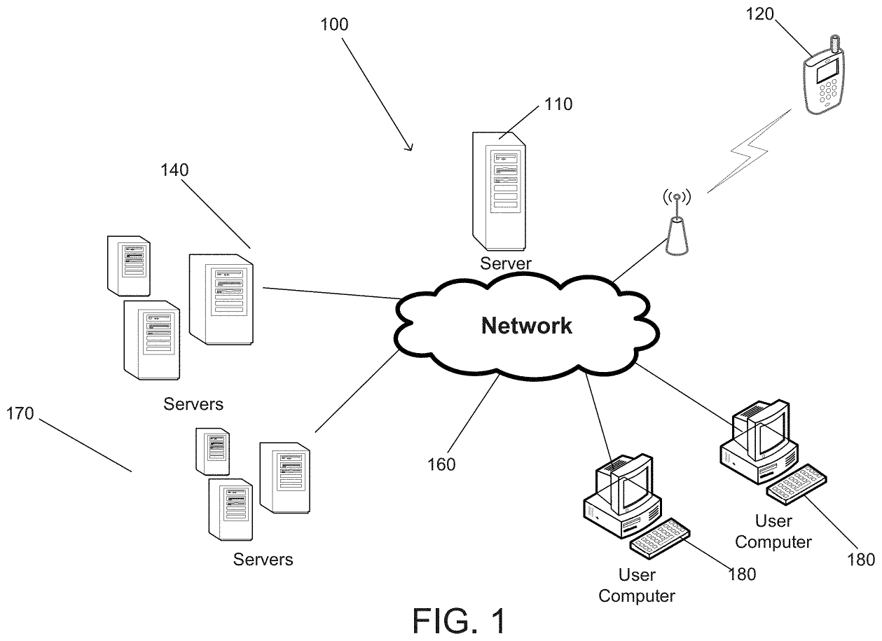

A vital sign measurement system capable of measuring vital signs in one or more patients in accordance with an embodiment of the invention is illustrated in FIG. 1. Network 100 includes a communications network 160. The communications network 160 is a network such as the Internet that allows devices connected to the network 160 to communicate with other connected devices. Server systems 110, 140, and 170 are connected to the network 160. Each of the server systems 110, 140, and 170 is a group of one or more servers communicatively connected to one another via internal networks that execute processes that provide cloud services to users over the network 160. The processes can include using multimodal data captured by different types of vital sign sensors in order to make a disease diagnosis. One skilled in the art will recognize that a vital signs measurement system may exclude certain components and/or include other components that are omitted for brevity without departing from this invention.

For purposes of this discussion, cloud services are one or more applications that are executed by one or more server systems to provide data and/or executable applications to devices over a network. The server systems 110, 140, and 170 are shown each having three servers in the internal network. However, the server systems 110, 140 and 170 may include any number of servers and any additional number of server systems may be connected to the network 160 to provide cloud services. In accordance with various embodiments of this invention, a vital signs measurement system that measures vital signs of one or more users/patients in accordance with an embodiment of the invention may be provided by a process being executed on a single server system and/or a group of server systems communicating over network 160.

Users may use personal devices 180 and 120 that connect to the network 160 to perform processes that measure vital signs in accordance with various embodiments of the invention. In the illustrated embodiment, the personal devices 180 are shown as desktop computers that are connected via a conventional “wired” connection to the network 160. However, the personal device 180 may be a desktop computer, a laptop computer, a smart television, security camera system, image capture device, or any other device that connects to the network 160 via a “wired” connection. The mobile device 120 connects to network 160 using a wireless connection. A wireless connection is a connection that uses Radio Frequency (RF) signals, Infrared signals, or any other form of wireless signaling to connect to the network 160. In FIG. 1, the mobile device 120 is a mobile telephone. However, mobile device 120 may be a mobile phone, Personal Digital Assistant (PDA), a tablet, a smartphone, or any other type of device that connects to network 160 via wireless connection without departing from this invention. The mobile device 120 and/or personal device 180 can include one or more cameras for capturing images, recording videos and also one or more sensors for capturing vital sign data. In many embodiments, mobile device 120 and/or personal device 180 can include 3D and/or 2D image sensors, audio sensors, among other vital sign sensors.

The mobile device 120 and/or personal device 180 can process the image data, video data, and/or vital sign data. The mobile device 120 and/or personal device 180 can process the sensor data using different transformer models. The mobile device 120 and/or personal device 180 can generate an output related to a disease diagnosis using the transformer models. In many embodiments, mobile device and/or personal device 180 can transmit the image data, video data, and/or sensor data to the one or more servers 110, 140, and 170 over the network 160 and the servers 110, 140, and 170 can process the data in order to make a determination regarding a disease diagnosis.

As can readily be appreciated the specific computing system used to capture image data and/or measure vital signs is largely dependent upon the requirements of a given application and should not be considered as limited to any specific computing system(s) implementation.

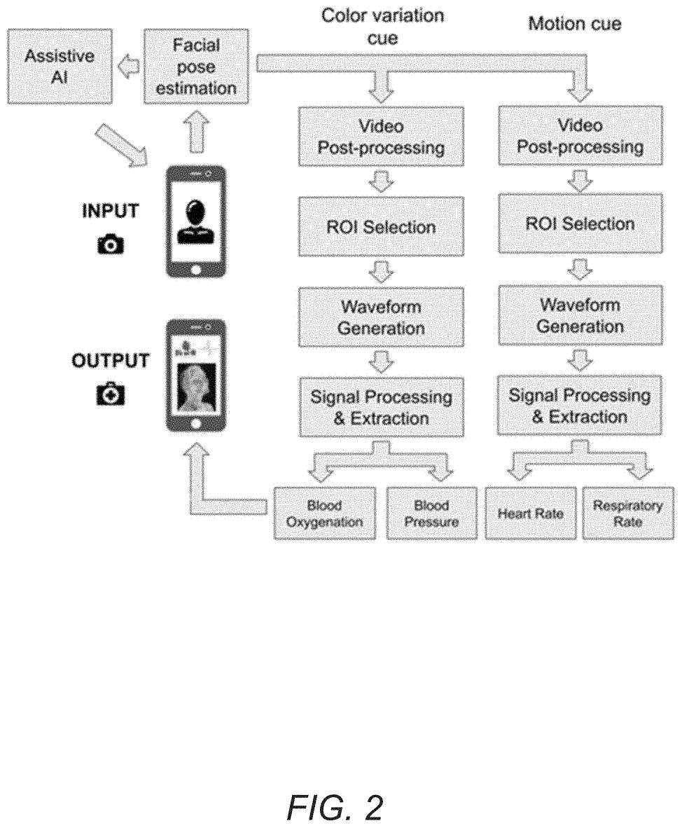

A vital sign measurement system in accordance with an embodiment of the invention is illustrated in FIG. 2. As illustrated, input video of a user is captured using a mobile device. While the illustrated embodiment utilizes video captured with a conventional camera, it should be readily appreciated that systems and methods in accordance with various embodiments of the invention can utilize multi-modal imaging systems to capture input data. Referring again to FIG. 2, the captured video data can be evaluated for facial pose estimation. In certain embodiments, assistive AI can be employed to provide directions to a user to improve the quality of the captured inputs. The captured inputs can then be processed in parallel pipelines for color variation cues and motion cues.

Each pipeline can perform video post-processing, region of interest (ROI) selection, waveform generation, and/or signal processing and extraction. Each of these pipelines can be similar, but may be adjusted to emphasize color variation and motion cues respectively. Processes for video post-processing, ROI selection, waveform generation, and signal processing and extraction are described in greater detail below.

The results of the color variation cue pipeline and the motion cue pipeline can be used to measure vital signs, such as (but not limited to) blood oxygenation, blood pressure, heart rate, and/or respiratory rate. The measured vitals can then be displayed for the user on the user's device. In a number of embodiments, measured vital signs can be further analyzed to screen for potential conditions and can provide notifications or alerts to a user to seek treatment.

Although many of the examples described herein describe measuring vital signs for individuals, one skilled in the art will recognize that similar systems and methods can be used in a variety of applications, including (but not limited to) measuring vitals for individuals in a crowd (e.g., via a surveillance system), and/or measuring vital signs of animals (e.g. individual animals and/or animals in a group/herd) without departing from the scope of the invention.

### Vital Sign Measurement Devices

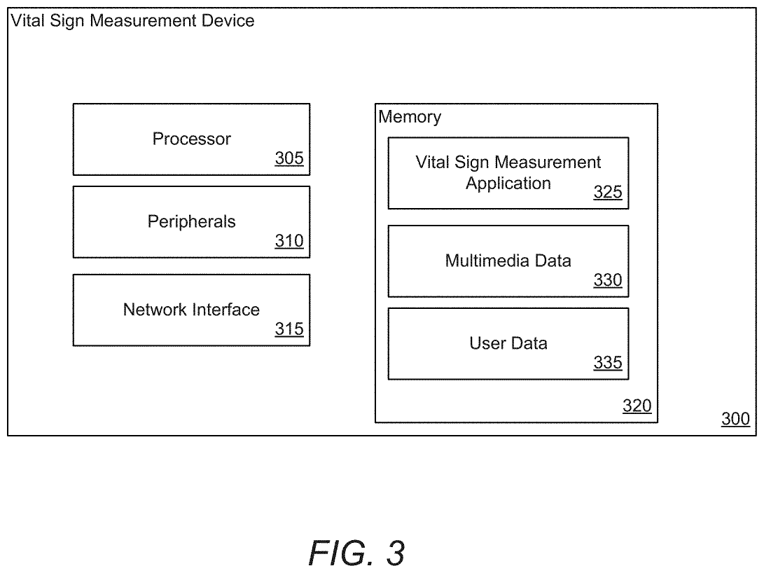

A vital sign measurement device that executes instructions to perform processes that measure vital signs in accordance with an embodiment of the invention is illustrated in FIG. 3. Vital sign measurement devices in accordance with many embodiments of the invention can include (but are not limited to) one or more of mobile devices, cameras, and/or computers. In many embodiments, the vital sign measurement device can include multiple sensors to enable multimodal sensing of signals. In certain embodiments, the multiple sensors can be integrated within the vital sign measurement device or provided as a peripheral sensor that can connect to a conventional computing device.

In certain embodiments, vital sign measurement device can be incorporated within surveillance systems and other image capture systems. Vital sign measurement element 300 includes a processor 305, peripherals 310, a network interface 315, and memory 320. One skilled in the art will recognize that a vital sign measurement element may exclude certain components and/or include other components that are omitted for brevity without departing from this invention.

The processor 305 can include (but is not limited to) a processor, microprocessor, controller, or a combination of processors, microprocessor, and/or controllers that execute instructions stored in the memory 320 to manipulate data stored in the memory. Processor instructions can configure the processor 305 to perform operations as appropriate to the requirements of specific applications in accordance with various embodiments of the invention.

Peripherals 310 can include any of a variety of components for capturing data, such as (but not limited to) cameras, displays, and/or sensors. In a variety of embodiments, peripherals can be used to gather inputs and/or provide outputs. Vital sign measurement element 300 can utilize network interface 315 to transmit and receive data over a network based upon the instructions performed by processor 305. Peripherals and/or network interfaces in accordance with many embodiments of the invention can be used to gather inputs that can be used to measure vital signs.

Memory 320 includes a vital sign measurement application 325, multimedia data 330, and model data 335. Vital sign measurement applications in accordance with several embodiments of the invention can be used to measure vitals. In some embodiments, vital sign measurement applications can be executed on standard smartphone hardware. In several embodiments, the image data captured by a standard smartphone camera can be processed using consumer-grade image signal processor (ISP) hardware. In a number of embodiments, the video processing components of the pipeline, namely video capture, motion detection and illumination correction are optimized as a single suite for efficient and fast execution on the smartphone hardware.

Multimedia data in accordance with a variety of embodiments of the invention can include various types of multimedia data that can be used in evaluation processes. In certain embodiments, multimedia data can include (but is not limited to) video, images, and/or audio. Multimedia data in accordance with some embodiments of the invention can include video captured by an individual to measure their vital signs to be transmitted to a physician for review. In some embodiments, multimedia data can include video captured of multiple individuals, where vital signs can be measured for one or more of the individuals in the video. As is discussed further below, specialized hardware can be utilized to capture additional sensor data including multi-modal image data. The specific sensor data that is acquired and the specific hardware utilized in the acquisition of the sensor data is largely dependent upon the requirements of particular applications.

In several embodiments, user data can store various information about a user, such as (but not limited) a history of previous vital sign measurements, medical record data, and/or personal information. User data in accordance with certain embodiments of the invention can protect a user's privacy by storing user identifiable information (e.g., name, images, etc.) separately from vital sign measurement data. In various embodiments, vital sign measurement data is transmitted to another device (e.g., a server, cloud service, etc.), but can be transmitted securely (e.g., separately from any user identifiable data, with anonymized identification data, etc.).

Although a specific example of a vital sign measurement element 300 is illustrated in FIG. 3, any of a variety of vital sign measurement device can be utilized to perform processes for measuring vital signs similar to those described herein as appropriate to the requirements of specific applications in accordance with embodiments of the invention.

### Vital Sign Measurement Application

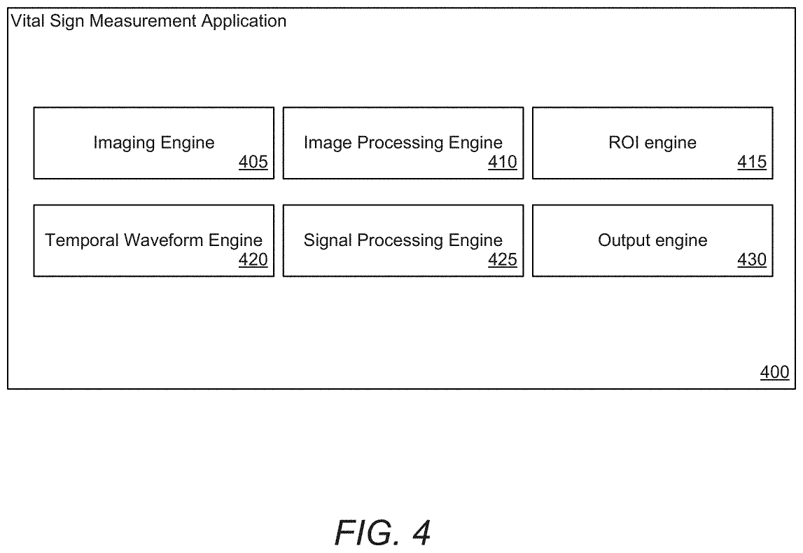

A vital sign measurement application for measuring vital signs of a user/patient in accordance with an embodiment of the invention is illustrated in FIG. 4. Vital sign measurement applications in accordance with several embodiments of the invention can operate as standalone applications running on a user's device. Alternatively, or conjunctively, vital sign measurement applications in accordance with many embodiments of the invention can operate as backend software, operating on a server or cloud service, that can be embedded as features in an existing application.

In the illustrated embodiment, the vital sign measurement application 400 includes imaging engine 405, image processing engine 410, ROI engine 415, temporal waveform engine 420, signal processing engine 425, and output engine 430. One skilled in the art will recognize that a vital sign measurement application may exclude certain components and/or include other components that are omitted for brevity without departing from this invention.

Imaging engines in accordance with some embodiments of the invention can be used to capture inputs for measuring vitals. Inputs in accordance with a number of embodiments of the invention can include (but are not limited to) facial video data, (e.g., captured while a subject is sitting down) or surveillance data (e.g., while subjects are walking around). Inputs in accordance with certain embodiments of the invention can be recorded over the course of several minutes to allow for variations due to subject motion and/or lighting fluctuations. To further ensure robustness, systems in accordance with a variety of embodiments of the invention can introduce a variety of illumination settings including, but not limited to, natural sun lighting, fluorescent tube lighting, and/or on-camera flash lighting. Systems in accordance with several embodiments of the invention can be configured to be robust to dropped frames and/or heterogeneity in video compression rates. In various embodiments, during video capture, systems in accordance with a variety of embodiments of the invention can provide an assistive AI unit that helps guide the subject towards recording a more favorable video that can provide more stable and accurate body vital sign estimates. Assistive AIs in accordance with some embodiments of the invention can receive information regarding facial features present in the scene and corresponding illumination variance over said features. Information regarding the spatial location of facial features with respect to the camera can be obtained using a real-time facial pose estimation algorithm in accordance with a number of embodiments of the invention. In several embodiments, from this information, a simple brightness variation metric can be calculated to determine the uniformity of the illumination of specific facial features of the subject. Assistive AIs in accordance with a number of embodiments of the invention can utilize such information to guide the subject to ensure a feasible orientation of the subject with respect to the camera and present illumination sources. Moreover, in many embodiments, this information can be passed to downstream components to enable accurate execution of tasks like motion stabilization and ROI selection.

Although many of the examples describe telemedicine in the context of regions of the face and shoulders, additional regions of the body could be scanned. For instance, in certain pathologies it is useful to obtain an estimate of oxygenation in different spatial locations (e.g., extremities, core body parts, etc.).

In a variety of embodiments, vital sign measurement can be performed for imaging of multiple people simultaneously. This can be useful for population-scale health monitoring. Consider, for instance, that a security camera in a populated location may image a million people a month. It may be desirable—in the midst of a pandemic for instance—to obtain vital signs of a population. Especially in such large-scale systems, vital sign measurement in accordance with some embodiments of the invention can implement privacy preserving policies by, for example, storing images/video of individuals separately and securely away from the measured vital signs.

In many embodiments, image processing engines can process captured video. After the video data is recorded, it can be desirable to pass it through a processing pipeline to improve the accuracy of vital sign estimates. In a number of embodiments, image processing can include (but is not limited to) motion stabilization and/or illumination normalization. In certain embodiments, image processing engines can process video for a color cue pipeline differently from video for a motion cue pipeline. For example, in applying video stabilization it is desirable that image processing engines, when acting on color cues, perform some stabilization. In contrast, the video stabilization may not be applied, or may be applied in a different way, if the means of extracting physiology include motion cues. Here, application of stabilization could corrupt the motion cues. Image processing engines in accordance with some embodiments of the invention can perform some processes equally between the different cue pipelines (e.g., illumination artifacts may be processed the same in response to either motion or color cues). In many embodiments, image processing engines can generate two output videos corresponding to each of the color and motion cues.

Motion stabilization in accordance with certain embodiments of the invention can implement specialized video stabilization processes for front-facing headshots, commonly referred to as “selfies”. Motion stabilization processes in accordance with many embodiments of the invention can utilize upstream information of spatial location of facial features information in combination with background motion information estimated using optical flow algorithms. Such approaches deviate from common stabilization techniques done by prior camera-based vital sign detection works, which instead use general algorithms that are not application-specific. In a variety of embodiments, motion stabilization, when detecting motion cues for associated body vital signs, can be directed towards removing large-scale motion associated with camera shake or voluntary subject motion. To do this, processes in accordance with numerous embodiments of the invention can mask out the foreground (e.g., subject's face) using upstream information from the spatial location of facial features. With the background now isolated, processes in accordance with many embodiments of the invention can use optical flow processes to track the motion of background features. This can be used to compensate for motion artifacts related to camera shake and other large motions.

In some embodiments, illumination normalization processes can be applied to each frame of the extracted videos. Illumination variance can be detrimental to approaches to extract vital signs. Such approaches to measuring pulse oxygenation can make use of the sub-surface reflectance and scattering, the extent of which depends on the blood oxygenation levels. However, it can be hard to obtain the subsurface scattering measurements due to a mixed direct light component to the image that arises due to external lighting. Illumination normalization in accordance with a number of embodiments of the invention can split an image into its global and direct light components, making use of the fact that the direct light component can be subtracted out to remove illumination variance for the extracted regions of interest. In numerous embodiments, the global component, which corresponds to sub-surface scattering, can then be further processed to extract the blood oxygenation levels of the subject.

Region of interest (ROI) engines in accordance with several embodiments of the invention can select ROIs from video. In several embodiments, ROIs can be selected from videos for the color and motion cues. ROIs in accordance with certain embodiments of the invention can include (but are not limited to) a subject's face, core, and/or extremities. Vital sign signals can be extracted from the selected ROIs. In a variety of embodiments, ROI selection can leverage a pre-trained convolutional neural network (CNN) architecture to segment facial skin frame-by-frame. After skin segmentation, adaptive weight sampling can be implemented for each body vital sign being detected. By using upstream information regarding brightness variation over specific facial features, downstream information regarding final output vital sign signal extraction, and a priori knowledge on how skin-tone complexion affects signal extraction, the weighted sampling strategy in accordance with many embodiments of the invention can be optimized to guarantee accuracy and robustness of the final vital sign being estimated. In numerous embodiments, hyperparameters of a segmentation CNN can be tuned with training data, which inherently requires training data to be optimized. Processes in accordance with many embodiments of the invention can ensure that training data is not biased in any way and contains a variety of subjects with differing skin tones, facial orientation, and illumination settings.

In a variety of embodiments, temporal waveform engines can generate temporal waveforms for selected ROIs from each body cue video. For the motion cue video, processes in accordance with some embodiments of the invention can generate waveforms by tracking the motion of facial features within the ROI between frames and calculating velocity vectors. For the color cue video, processes in accordance with many embodiments of the invention can generate waveforms by calculating differences in pixel values within the ROI between frames.

Signal processing engines in accordance with a variety of embodiments of the invention can to extract key body vital signs from the generated temporal waveforms. In various embodiments, signal processing engines can use component analysis and/or frequency filtering to isolate signals from the generated waveforms. Signal processing engines in accordance with many embodiments of the invention can perform isolation and post-processing based on the key vital sign being measured. In several embodiments, heartbeat can be estimated by examining motion cues within the head and neck regions. Respiratory rate in accordance with a variety of embodiments of the invention can be estimated by examining motion cues around the neck and shoulder regions. In a variety of embodiments, blood oxygenation can be estimated by examining color cues between the red and green color channels in the head regions. Blood pressure can be estimated by examining color cues across all color channels in the head and neck region. While the above represents the extraction of key vital signs, there are additional vital signs that can be considered in a similar fashion.

Output engines in accordance with several embodiments of the invention can provide a variety of outputs to a user, including (but not limited to) charts, images, vital sign data, notifications, instructions, and/or alerts. In numerous embodiments, output engines can communicate with other devices to transmit vital sign data. Although names of patients can be anonymized, the capture of facial data is identifiable data. In certain embodiments, privacy can be preserved by having the vital sign data be stored separately from the identifiable image data.

Although a specific example of a vital measurement application 400 is illustrated in FIG. 4, any of a variety of vital sign measurement applications can be utilized to perform processes for measuring vital signs similar to those described herein as appropriate to the requirements of specific applications in accordance with embodiments of the invention.

### Processes for Measuring Vital Signs

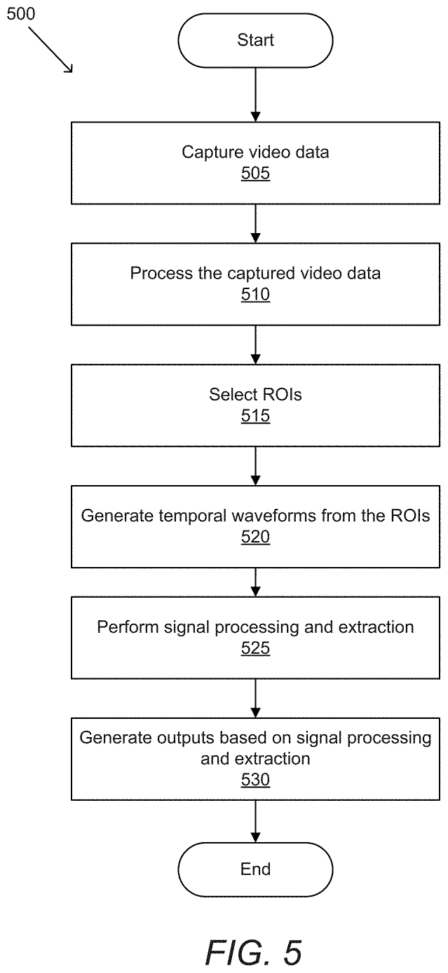

A process for measuring vital signs in accordance with an embodiment of the invention is illustrated in FIG. 5. Process 500 captures (505) video data. In several embodiments, video data can include (but is not limited to) video of a person's face, upper body, full body, among others. While the discussion that follows with respect to FIG. 5 illustrates a single video feed, system and methods in accordance with many embodiments of the invention can take multiple sensor modalities that include a video feed, image data, audio data, and/or more vital sign sensor data as inputs.

In several embodiments, capturing the video data can include capturing video, analyzing the video and/or estimated characteristics (e.g., facial pose, nuisance levels, etc.) from the video using an assistive AI, and providing feedback to capture additional video data. Feedback in accordance with many embodiments of the invention can include instructions for changes to the capture, alerts that a capture is not sufficient, etc.

Process 500 processes (510) the captured video data. Processing the video data in accordance with several embodiments of the invention can include generating new videos. In numerous embodiments, processing the videos can include (but is not limited to) performing motion stabilization, illumination normalization, sharpening, and/or contrast enhancement. In a number of embodiments, multiple cue copies of the captured video are created to analyze different cues (e.g., motion cues, color cues, etc.). Cue copies of the video in accordance with numerous embodiments of the invention can be processed differently, depending on the cue that is being analyzed. For example, video for color cues may be motion stabilized, while video for motion cues may not apply motion stabilization at all or may apply a modified motion stabilization.

Process 500 can select (515) regions of interest (ROIs). ROIs in accordance with certain embodiments of the invention can include (but are not limited to) a subject's face, core, and/or extremities, from which an individual's vital signs can be extracted. Selecting ROIs in accordance with many embodiments of the invention can be performed using a neural network (e.g., a segmentation CNN) to identify target areas of each frame of a video. Target area selection in accordance with some embodiments of the invention can be based on various characteristics of portions of the video frames, such as (but not limited to) texture, motion, relative spatial location (e.g., particular locations of a person's face), etc. ROI selection in accordance with numerous embodiments of the invention can be performed differently for different cue videos. In some embodiments, adaptive weight sampling can be implemented for each vital sign being detected. In several embodiments, different weights can be applied for different vital signs even within the same cue video.

Process 500 can generate (520) temporal waveforms from the regions of interest. Generating temporal waveforms for motion cue video in accordance with a number of embodiments of the invention can include tracking motion of facial features with the ROI between frames and calculating velocity vectors. In a variety of embodiments, waveforms for color cue video can be generated by calculating differences in pixel values within the ROI between frames of the video.

Process 500 can perform (525) processes for signal processing and extraction. Signal processing and extraction in accordance with a number of embodiments of the invention can be used to extract vital signs from the generated temporal waveforms. In a variety of embodiments, signal processing can include component analysis and/or frequency filtering to identify patterns in the signals, such as (but not limited to changes in colors (or color channels), periodic motion in the head and neck region from the motion cue video, etc.

Process 500 can generate (530) an output based on the signal processing and extraction. Outputs in accordance with numerous embodiments of the invention can include visual data (e.g., charts, images, etc.), notifications (e.g., alerts, reminders, warnings, etc.) and/or text data (e.g., vital sign data, instructions, recommendations, among others). In various embodiments, the generated outputs can be transmitted to a physician for a review or as part of a telemedicine session. Generating outputs in accordance with various embodiments of the invention can include anonymizing and otherwise protecting the data.

While specific processes for measuring vital signs are described above, any of a variety of processes can be utilized to measure vitals as appropriate to the requirements of specific applications including (but not limited to) using specialized hardware and/or sensing data in multiple imaging modalities. In certain embodiments, steps may be executed or performed in any order or sequence not limited to the order and sequence shown and described. In a number of embodiments, some steps may be executed or performed substantially simultaneously where appropriate or in parallel to reduce latency and processing times. In some embodiments, one or more of the above steps may be omitted. A variety of systems and methods that utilize multimodal sensor data in accordance with various embodiments of the invention are discussed further below.

### Multimodal Health Sensing Platform

Many embodiments provide for a multimodal health (MMHealth) sensor platform for remote diagnosis of various medical conditions. In many embodiments, the MMHealth platform can include (but are not limited to) 3D and/or 2D image sensors, at various spectral bandwidths, along with mixed modalities such as radio frequency and acoustic chipsets. The hardware in accordance with many embodiments can be tightly coupled with multimodal inference software which can aggregate data, using both parametric and neural network compute. In many embodiments, the multimodal platform can be applied to a variety of different applications, including include infectious disease diagnosis (e.g., COVID-19, other viral and bacterial pneumonias), mental health disorders (e.g., via ocular cues, facial expression cues), sleep disorders, heart arrhythmias, stroke, among numerous other applications.

Remote diagnosis of infectious diseases, such as COVID-19 or other pneumonias, can offer a safe and quick way of evaluating and triaging the level of medical attention and care required by an individual. In light of changing environments and the recent COVID-19 pandemic, it would be beneficial to provide a non-contact method that suggests an infectious disease diagnosis in the event that contact with the skin or nasal region for a laboratory sample is either infeasible or should be avoided for medical reasons. Such contact-less assessment and diagnosis using a multimodal health sensing platform would especially be beneficial in the outpatient setting, where clinicians make a clinical diagnosis of an outpatient pneumonia using patient history, subjective symptoms, and physical examination findings and typically do not order laboratory tests for diagnosis. The MMHealth platform in accordance with many embodiments of the invention may assist in clinical evaluations to more accurately quantify and suggest a likely infectious disease diagnosis to a physician in a non-contact manner.

Improvements in remote photoplethysmography (r-PPG), respiratory rate and privacy enabled motion tracking have enabled non-contact vital sign measurements such as heart rate (HR), heart rate variability (HRV), blood volume pulse (BVP), breathing rate, breathing rate variability among others. This information can be clinically acquired to assist physicians in diagnosing an infectious disease in patients. Accordingly, many embodiments of the multimodal health platform provide an end-to-end system that includes a multimodal sensor stack with networking capabilities for remote disease diagnosis. In many embodiments, this can allow for rapid testing at scale by streamlining privacy-enabled data collection methods and fast tracking the data mining search for models capable of identifying infectious disease signatures.

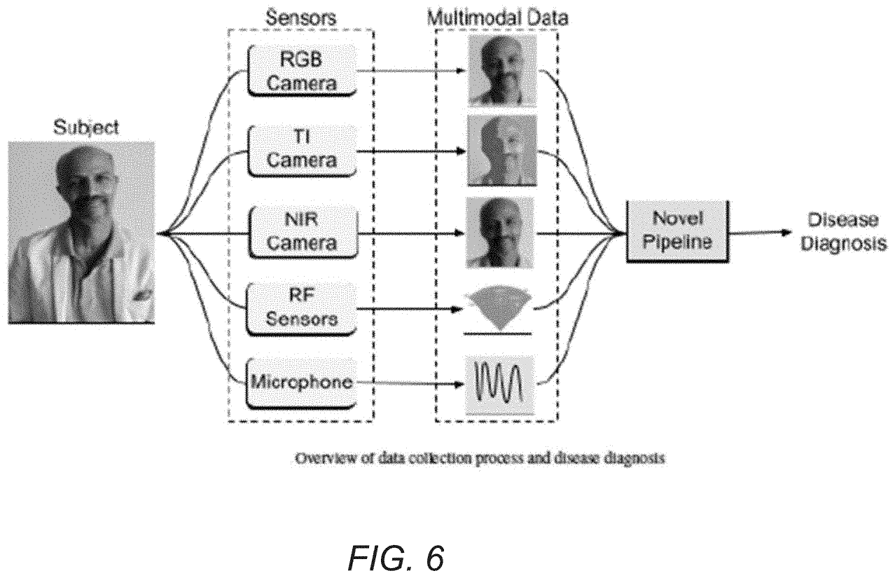

Many embodiments of the system provide a multimodal end-to-end pipeline for remote diagnosis of infectious diseases. In several embodiments, the system may use multiple modalities for estimating different vital sign measurements such as individual heart rates, where the addition of the multiple modalities improves classification performance. FIG. 6 illustrates an overview of data collection process and disease diagnosis in accordance with an embodiment of the invention. The sensors can include an RGB camera, TI camera, NIR camera, RF sensors, microphone. The multimodal data can be processed to obtain different types of data for different sensors. The data from the different sensors can be used in combination to provide a disease diagnosis. Although FIG. 6 illustrates a particular data collection process for disease diagnosis using a particular set of sensors, any of a variety of sensors can be used as appropriate to the requirements of specific applications in accordance with embodiments of the invention.

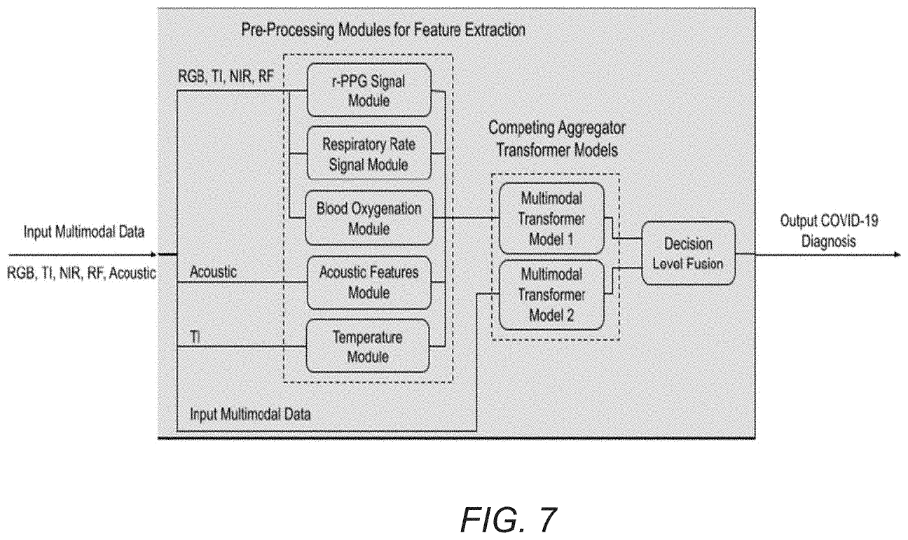

In a number of embodiments, the multimodal data can be used to generate robust r-PPG signals, respiratory rate and blood oxygenation features. These three features, along with the unimodal and data-mined features, can then be used to train multimodal transformer models and generate a final infectious disease diagnosis. FIG. 7 illustrates a COVID-19 diagnosis multimodal pipeline in accordance with an embodiment of the invention. In particular, the multimodal pipeline can receive input multimodal data, including RGB, TI, NIR, RF, and/or acoustic data. The input multimodal data can be pre-processed using different pipelines for feature extraction, including (but not limited to) an r-PPG signal pipeline, a respiratory rate signal pipeline, a blood oxygenation pipeline, an acoustic features pipeline, and/or a temperature pipeline. This data can be processed using competing aggregator transformer models, including a multimodal transformer model 1 and a multimodal transformer model 2. This data can be provided for decision level fusion and to provide a diagnosis (e.g., output COVID-19 diagnosis). Although FIG. 7 illustrates a particular multimodal pipeline that includes data from a set of sensors, any of a variety of sensors and pipelines can be utilized as appropriate to the requirements of specific applications in accordance with embodiments of the invention.

r-PPG Pipeline

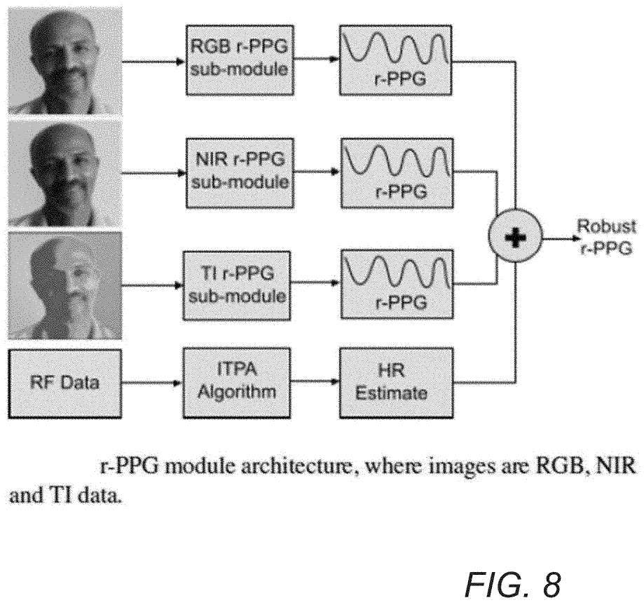

In many embodiments, different methods can be used to obtain a final r-PPG signal. FIG. 8 illustrates two methods of obtaining a final r-PPG signal, where images are RGB, NIR, and TI data in accordance with an embodiment of the invention. In many embodiments, both methods may require pre-processing the RGB to remove specular components of the reflected light from the image. In addition, the thermal images can be pre-processed using a face detection CNN to zoom into the carotid arteriovenous complex on the neck and superficial temporal vessel complex on the face of the patient. Finally, in both methods, the HR estimate can be obtained from the RF phased array system using two stage beam-steering and an interferometric time-phase analysis (ITPA) process. This estimate can then be aggregated with the r-PPG estimate from either method to obtain the final robust r-PGG estimate.

In a first method in accordance with certain embodiments, a multimodal transformer can be used to train on the pre-processed inputs to obtain the r-PPG estimate. In certain embodiments, two modified multimodal models can be tested; a UniT transformer and a ViT transformer. Advantages can include that patterns and correlations can be learned and identified between both the visual and thermal images throughout the model, and the end-to-end method can improve benchmark performance in multiple computer vision fields.

In a second method embodiments in accordance with certain embodiments, an r-PPG estimate can be obtained from the RGB, NIR and TI data separately, and then aggregated together. PCA can then be applied to these three r-PPG signals, where the first component with the greatest variance is chosen as the final r-PPG signal. The benefit can include that the individual r-PPG estimates can be robust. Certain limitations can include that by individually aggregating the resulting signals, there is a risk of loss of information, error propagation and no learning occurring between the two input RGB and TI data streams.

RGB r-PPG: In many embodiments, the r-PPG estimate from RGB data can be obtained by using different techniques as appropriate to the requirements of different applications in accordance with embodiments of the invention.

NIR r-PPG: In many embodiments, the r-PPG estimate from TI data can be obtained by expanding certain known techniques to 3D images to combat motion noise. Certain embodiments can identify the facial landmarks, process the image to get a raw r-PPG matrix and denoise via Robust PCA to obtain a low-rank matrix. The final signal can be obtained by further denoising by elucidating the sparse frequency signal corresponding to the r-PPG waveform.

TI r-PPG: In many embodiments, the r-PPG estimate from TI data can be obtained using certain techniques as appropriate to the requirements of different applications in accordance with embodiments of the invention. Although FIG. 8 illustrates an r-PPG pipeline architecture, where images are RGB, NIR, and TI data, any of a variety of pipeline architecture that include images from different types of cameras and/or sensors can be utilized as appropriate to the requirements of specific applications in accordance with embodiments of the invention.

### Respiratory Rate Pipeline

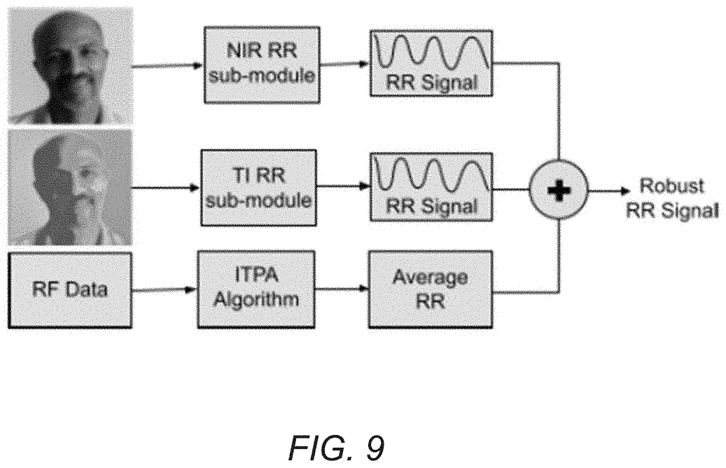

Different methods can be used for obtaining the final respiratory waveform signal. FIG. 9 illustrates two methods that can be used for obtaining a final respiratory waveform in accordance with an embodiment of the invention. In particular, FIG. 9 illustrates a respiratory rate pipeline architecture, where images are NIR and TI data. In many embodiments, an RR estimate is obtained from the RF phased array system using two stage beam-steering and an interferometric time-phase analysis (ITPA) processes. This estimate can then be aggregated with the respiratory waveform estimate to obtain the final robust respiratory estimate.

In certain embodiments, a multimodal transformer can be used to train on these pre-processed inputs to obtain the respiratory estimate. Two modified multimodal models can be tested; a UniT transformer and a ViT transformer. The advantage of this method is that patterns and correlations can be learned and identified between both the visual and thermal images throughout the model, and this end-to-end method can improve benchmark performance in multiple computer vision fields.

In certain embodiments, a respiratory waveform estimate can be obtained from the NIR and TI data separately, and then aggregated together. PCA can then be applied to these two respiratory signals, where the first component with the greatest variance can be chosen as the final respiratory signal.

NIR respiratory waveform: In many embodiments, the respiratory waveform estimate from NIR data can be obtained by using certain known techniques as appropriate to the requirements of different applications in accordance with embodiments of the invention.

TI respiratory waveform: In many embodiments, the respiratory estimate from TI data can be obtained using certain known techniques as appropriate to the requirements of different applications in accordance with embodiments of the invention. The 3D RGB model can be specifically used to map the identified feature landmarks to the TI data.

### Blood Oxygenation Pipeline

Certain known techniques can be used for the blood oxygenation pipeline as appropriate to the requirements of different applications in accordance with embodiments of the invention. The band-limited amplified skin reflectance variations at the 3D forehead ROI points can be added to their original grey value variations, resulting in enhanced NIR grey value signals. At each time point, the spatial standard deviation and mean value of the enhanced NIR grey values within the forehead ROI are calculated as AC and DC value. The AC-to-DC ratio R can then be calculated:

$${R}_{λ} = \frac{A {C}_{λ}}{D {C}_{λ}}$$

In the end, the ratio of ratios RR, which should be linear to the SₚO₂ value, can be calculated by calculating the AC and DC components at both 780 and 940 nm. In order to improve the stability of oxygen saturation estimation, the RR values should be averaged over a certain time window.

$$R R = \frac{{R}_{780}}{{R}_{940}} = \frac{A {C}_{780} D {C}_{940}}{A {C}_{940} D {C}_{780}}$$

### Unimodal Feature Extraction

In many embodiments, individual unimodal data can be used to generate acoustic and temperature features from forced coughs and speech. These three features, along with the multimodal and data-mined features, can then be used to train the multimodal transformer models and generate a suggestion of a likely infectious disease diagnosis.

### Acoustic Features Pipeline

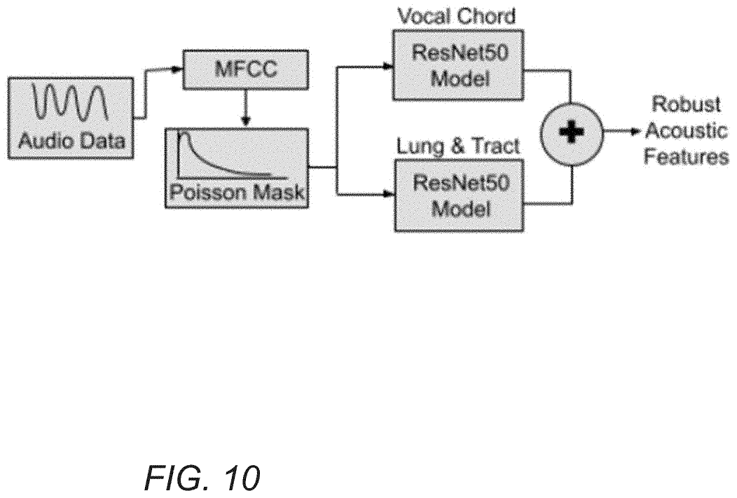

In many embodiments, the acoustic data is divided into two sections: one for continuous speech and one for forced coughs. The former can be used to train a pre-trained model to learn vocal cord features and the latter can also be used to train a pre-trained model, but to learn features inherent to the state of the respiratory tract and lungs. As pre-processing, both divisions of the dataset can be trimmed into a number of second window (e.g., 6 second windows), padded as required, and the MFCC values are calculated. A Poisson Mask can be applied to introduce muscle fatigue and degradation features. The data can then be fed into the corresponding model. An acoustic features pipeline architecture in accordance with an embodiment of the invention is illustrate in FIG. 10. In particular, FIG. 10 illustrates audio data with MFCC values calculated, with a Poisson Mask applied and the data fed into a vocal chord model and a lung and tract model.

In many embodiments, the Poisson mask applied to both the train and test sets is shown in Equation 1 below, and can follow from known memory decay models.

The Poisson Mask applied to a specific MFCC value Ix can be calculated by multiplying this value by a random Poisson distribution of parameters Ix and λ, where λ is the average value of the entire MFCC set. Although FIG. 10 illustrates a particular acoustic features pipeline architecture that includes two models, any of a variety of architecture that include different masks and models can be utilized as appropriate to the requirements of specific applications in accordance with embodiments of the invention.

$$M ( {I}_{x} ) = P o i s s ( λ ) {I}_{x} \qquad ( l )$$

$$Poiss ( X = k ) = \frac{{λ}^{k} {exp}^{- k}}{k !}$$

### Temperature Pipeline

Based on the RGB image data, many embodiments can use a model to detect the facial landmarks based on Local Binary Features (LBF). The inner eye corner regions can be used for body temperature estimation as they are less likely to be influenced by ambient airflow. These points can then be mapped to the TI data, and the temperature is estimated.

### Decision Level Fusion

In many embodiments, a final pipeline of an MMH health pipeline can include one or more multimodal transformer models. In certain embodiments, the final pipeline can include two competing aggregator multimodal transformer models. A first model can accept as inputs the vital sign and acoustic features obtained from the pre-processing pipelines, for example the three pre-processing pipelines, while a second model can accept as input the raw multimodal data captured from the sensors without any processing. Both of these models can then output an infectious disease diagnosis, (e.g., infectious disease diagnosis such as a COVID-19, among many others).

Many embodiments provide a fusion architecture where two separate multimodal transformers can be combined using decision level fusion to classify the data. In many embodiments, different methods can be used to combine the classifiers and perform data fusion as appropriate to the requirements of specific applications in accordance with embodiments of the invention.

Fuzzy Aggregation: The fuzzy integral is one operator in fuzzy set theory for fuzing different types of information. It is a generator function and produces many other well-known fuzzy operators such as ordered weighted averaging (OWA). The fuzzy integral can take on many different forms depending on the fuzzy measure g. The discrete Choquet Integral (CI) is:

C_{g}^{j}(d)=Σₜ₌₁^{T}d_{((t),j)}(x)[g(Aₜ)−g(Aₜ₋₁)],

where C_{g}^{j}(d) is the integral for class j and fuzzy measure g, the inputs are sorted in decreasing order, and Aₜ is the set of inputs from (1) to (t). Per class, the CI can be used to fuse the classifier inputs, and the highest confident class is then selected. The CI can be learned using known quadratic convex optimization processes and algorithms. Certain embodiments can test the known Linear Order Statistic Neuron (LOSN) method for decision-level fusion in place of the CI, which can have an exponentially growing number of parameters as a function of the number of inputs. Fuzzy aggregation can have advantages, including working well with incomplete information from each modality and providing a transparent method for aggregation. The LOSN can learn the best combination rule for a given application, and an analysis of its weights can provide interpretable results and thus can be considered a type of explainable AI.

Described below are physical setups and constructions of multi-modal health (MMHealth) sensor stack devices in accordance with many embodiments of the invention and details on curated datasets.

### Physical Imaging Sensors Implementation

MMHealth devices in accordance with several embodiments can be built on an optical breadboard that captures sensing streams with high fidelity. Sensors can include a color RGB camera, one or more processing chipsets, one or more mmWave sensors, one or more thermal cameras, one or more NIR cameras (e.g., two NIR cameras, one at 780 nm and one at 940 nm), one or more microphones. In many embodiments, a frame rate of 30 Hz can be realized for each of the 3D camera sensors, and they can be synchronized. In many embodiments, the entire camera system can exhibit a lateral measurement field of approximately 500 mm×400 mm at a distance of 1.5 m.

In many embodiments, a 3D sensor that includes two high speed cameras with a frame rate of 300 Hz in stereo arrangement and a high-speed projector. The 3D sensor can work at 850 nm and is therefore irritation-free. Using a set of (e.g., 10) projected light patterns, a 3D point cloud can be calculated using known stereo image correlation methods with known acceleration algorithms. With the use of a Graphics Processing Unit (GPU), a 3D frame rate up to 30 Hz can be realized. The high-speed cameras of the stereo-vision setup and both NIR cameras can be equipped with band-pass optical filters with respective central wavelengths and a full-width at half-maximum (FWHM) of 50 nm. The projector can include a band-pass filter (e.g., at 850 nm with a 50 nm FWHM) in order to avoid possible spectral crosstalk between pattern projection and NIR cameras.

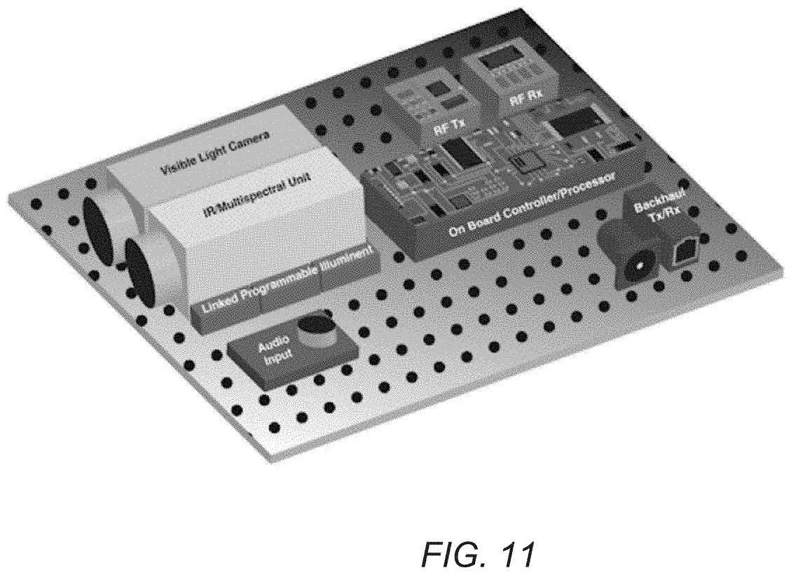

In many embodiments, a light emitting diode (LED) array can be included for homogenous lighting, with one LED at e.g., 780 nm and three LEDs at e.g., 940 nm. The beam angle of each LED can be between a certain number of degrees, e.g., between 90° and 120°, and its power can be e.g., 1 W. In certain embodiments, the total irradiation at a distance of 1.5 m can be about 1.255 μW/mm2, and therefore within eye safety limits. Sensor functionality can be verified using an onboard processor that runs in-built testing scripts. In many embodiments, latency from the processor to on-premise compute can be less than 100 ms, and read/write requests can be completed within 400 ms. In the event of network failure, an amount of sensor data (e.g., 30 GB of sensor data) can be stored without interruption, which is equivalent to a number (e.g., 100) of subjects. FIG. 11 illustrates a schematic of the multi-modal health sensor stack in accordance with an embodiment of the invention. As illustrated in FIG. 11, the multi-modal health sensor stack can be mounted on an optical slab. The multi-modal health sensor stack can include a visible light camera, an IR/multispectral unit, and audio input, RF Tx, RF Rx, on board controller/processor and a backhaul tx/rx. Although FIG. 11 illustrates a particular multi-modal health sensor stack architecture that includes a particular set of sensors and cameras, any of a variety of sensors, cameras, including RGB, NIR, among others and data inputs can be utilized as appropriate to the requirements of specific applications in accordance with embodiments of the invention.

In many embodiments, the multimodal 3D imaging system can be calibrated in order to obtain both intrinsic and extrinsic parameters of the cameras using known methods. 2D and 3D image data from different imaging devices can then be pixel-synchronously aligned with each other and combined into multimodal video frames. In certain embodiments, the resulting multimodal 3D video can have a lateral resolution of e.g., 750×600 pixels. The 3D point cloud of a subject's face can be reconstructed in high quality and high resolution despite some gaps in the hair region due to low reflectance, and the textures in different image modalities can be reasonably mapped onto the 3D point cloud without any visible alignment errors. The high-quality 3D image data in the face region ensure an accurate and robust 3D face tracking, and the fine texture mapping indicates a precise superimposition of heterogeneous multispectral and thermal image data and, thus, enables an effective analysis of these multimodal image data for vital sign estimation.

### Physical RF Sensor Implementation

In certain embodiments, three different RF bands can be used for vital sign detection, which include a millimeter Wave (mmWave) sensor, X-band custom CMOS chipset and an ISM band sensor. These sensors can support a number (e.g., 4) elements phased arrays which can provide high directionality and angular resolution. Many embodiments can use mmWave RF sensors. Certain embodiments may use known FMCW mmWave techniques, CMOS-enabled X-band RF techniques and associated ITPA algorithms.

In many embodiments, the system can use mmWave FMCW radars that provide for discriminating range and localizing, which can allow for multi-subject vital signs detection as the radar can differentiate reflections from different ranges. In certain embodiments, the FMCW radars' high propagation attenuation can reduce the probability of echo signals that bounce off multiple reflectors. FMCW radars may also be robust to thermal noise.

In many embodiments, the mmWave sensor can operate from 76-81 GHz with a built-in DSP and ARM processor for post-processing. In many embodiments, the collected data can be transferred through the networking pipeline for signal analysis and post-processing. In many embodiments, at e.g., 77 Ghz, λₘₐₓ is 3.9 mm, which allows for the detection of subtle changes in the distance between the subject and the radar. In the average adult, the chest moves 1-12 mm due to breathing and 0.01-0.05 mm due to heartbeat. In many embodiments, the mmWave sensor can have e.g., 3 transmitters and 4 receivers. In certain embodiments, the system can use only a single Tx/Rx pair. Each chirp can be e.g., 64 μs in duration, with an initial idle time of e.g., 7 μs, which may be required to settle the FMCW generator as it transitions from the end frequency to the start frequency. Therefore, e.g., Tᵣ=57 μs, and the slope of each chirp, K, is 70 MHz/μs. e.g. the seeping bandwidth is 3.99 GHz. Each chirp can be repeated every e.g., T_{c}=50 ms. With e.g., M=256, the observation window for both heart rate and respiratory rate becomes 12.8 s. The time resolution can be improved to 6.4 s by taking half the samples from the previous observation interval for the Doppler FFT, so that observation windows are overlapped with M/2 samples.

Many embodiments can use known ITPA algorithms as well as the underlying methods for estimation of the center and radius of a cloud point, phase unwrapping, and optimality of frequency estimation.

### Networking Pipeline and Cloud Compute

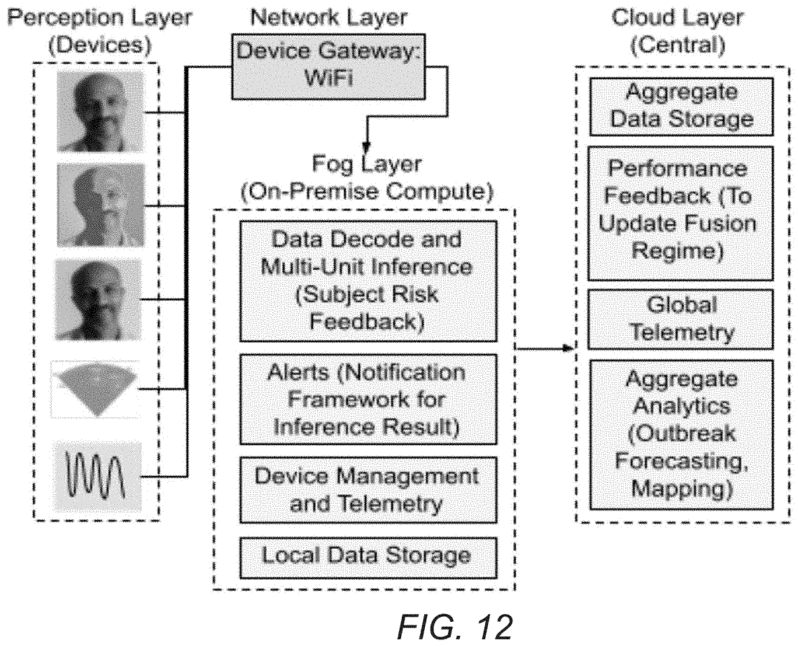

In many embodiments, a WiFi pipeline can be used to upload data collected by the MMHealth sensor stack to a secure network with cloud compute capabilities, including GPUs to pre-process the data, including mapping the 2D multi-modal image data to 3D for motion robustness. Cloud compute can also be used to process the multimodal data in the pipeline. In many embodiments, an IoT network architecture including a perception layer, network layer, fog layer, and cloud layer can be used to support scalability of the sensing platform. FIG. 12 illustrates a multi-modal health IoT network architecture for cloud compute in accordance with an embodiment of the invention. As illustrated in FIG. 12, the IoT network architecture can include perception layer, network layer, fog layer, and cloud layer. The network layer can include a device gateway (e.g., WiFi). The fog layer can include data decode and multi-unit inference pipeline, alerts (e.g., notification for inference result), device management and telemetry, and local data storage. The cloud layer can include an aggregate data storage, performance feedback (e.g,. to update fusion regime), global telemetry pipelines, and aggregate analytics (e.g., outbreak forecasting, mapping, among others).

In many embodiments, the perception layer includes on-device processing of sensor data utilizing an ARM processor for efficient programming and to reduce network load. The network layer can act as the sensor gateway supporting existing edge computing and WiFi, and Ethernet networking protocols. The fog layer can include on-premise compute that processes perception layer sensor data and locally stores and produces subject wise risk outputs. Premise device telemetry can also be monitored by the fog layer. In many embodiments, a fog Layer may enable, for example, each facility to track the traffic in and out of the building with the corresponding risk label. The cloud layer can manage data streams from each Fog layer for performance feedback to update fusion regime that is redeployed at the fog layer. In certain embodiments, aggregate analytics can be used to conduct global forecasting and mapping of vital signs and disease cases in real time. Although FIG. 12 illustrates a particular multi-modal health IoT network architecture for cloud computing, any of a variety of architectures for cloud computing can be utilized as appropriate to the requirements of specific applications in accordance with embodiments of the invention.

### 3D ROI and Facial Landmark Detection

Many embodiments may include 3D ROI and Facial Landmark Detection algorithms and processes. In many embodiments, front face detection is performed in the RGB image of the first frame. Several ROIs can be selected from the facial region for vital sign estimation. At the same time, feature points can be detected as preparation for the face tracking in the following video frames. In many embodiments, the ROI and feature points can be mapped to a 3D space using the camera parameters obtained from camera calibration. From the second video frame, the face can be tracked based on the detected features in 3D space, so the ROI can always be located at the same places on the face in each further frame. In these ROIs, different biosignals can be extracted and processed.

In the RGB image of the first frame, the frontal face and its eye regions can be detected using known detectors. Facial landmarks of a model can also be detected based on local binary features (LBF). In certain embodiments, different models for facial landmark detection can be used for face and eye detection. In many embodiments, three ROIs can be defined on the forehead, below the nostrils, and at the inner eye corners.

2D feature points can be used for face tracking and motion robustness, which are detected in the face region using known methods. In a feature point detection, a previously detected eye regions can be excluded from the face windows, due to involuntary eye motion. In many embodiments, the ROI and feature points can be mapped to a 3D space, so that 3D ROIs as 3D point clouds, as well as a set of 3D feature points, are generated.

### 3D Face Tracking Algorithm

Many embodiments of the system can use different face tracking algorithms. In many embodiments, once 2D feature points are detected in a first video frame, a tracker can be used from the second frame onwards. Then, the newly located feature points can be mapped to a 3D space, so that corresponding 3D point pairs between the 3D face in the current frame and the face in the first frame can be created. Under the assumption of few variations in facial expression, a 3D rigid body transformation with a certain number (e.g., 6) degrees of freedom (DoF) can be estimated from these point correspondences for the modeling of the current 3D head pose relating to the 3D face pose in the first frame. In many embodiments, a Kalman filter can be used to continuously correct the calculated face poses. Strong head motions can be recognized by calculating the temporal standard deviations of the elements of the 3D head pose (e.g., translation and rotation) within a number of frames (e.g., 100) and comparing them to a set of predefined thresholds. In many embodiments, if detected, the face tracking is terminated, and the next video frame is indexed with number 1 again.

In each valid video frame, the 3D ROIs generated in a first frame can be registered with the face in the current video frame by mapping them to the local 3D coordinate system of the current frame using the head pose corrected via Kalman filter. In many embodiments, these ROIs are steadily and exactly located at the same local positions on the face, even if strong head rotations occur. In this way, the continuous measurement of the same skin regions despite the head rotations can be realized.

### Training Dataset

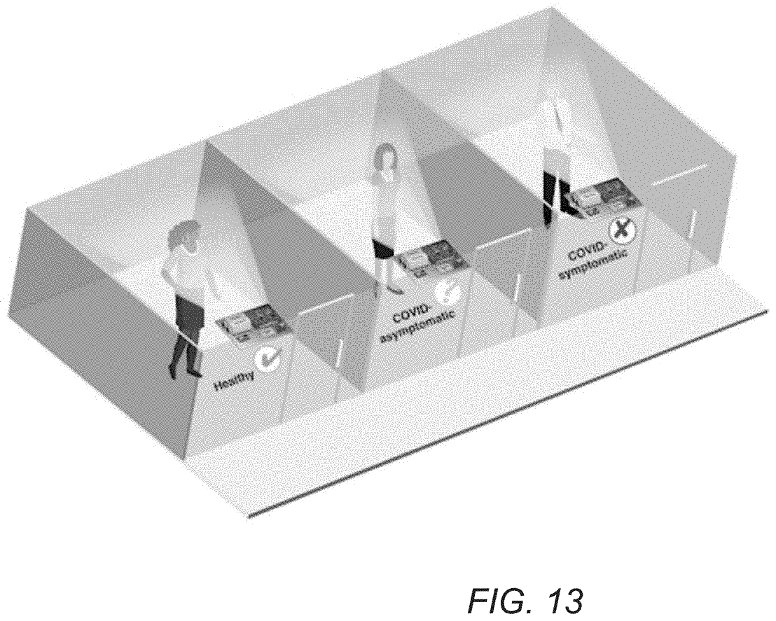

FIG. 13. Illustrates a separate, controlled environment that can be used for data collection in accordance with an embodiment of the invention. Per subject, the pre-screening dataset can include audio recordings that match video length (e.g. less than 60 seconds), RF Imaging video to monitor respiratory rate, visible light RGB video to detect r-PPG signal, and thermal imaging video to monitor body temperature, and also used to determine r-PPG and respiratory rate. Although FIG. 13 illustrates a particular setup of a separate, controlled environment for data collection, any of a variety of setups that provide for controlled environments can be utilized as appropriate to the requirements of specific applications in accordance with embodiments of the invention.

### Ground Truth Datasets

Many embodiments of the system provide for techniques in the context of ground truth datasets. In many embodiments, to capture ground truth for subjects, both PCR and Lateral Flow tests can be used to reduce false positive and false negative rates. Vital sign feature extraction of the r-PPG and respiratory rate signals can also be ground truthed against vital signs obtained via the vital sign monitor. These ground truth datasets can be used as training labels for feature extraction and final infectious disease diagnosis. The ground truth dataset can also be labelled by the age, sex, region, and race of the subject, as well as date and outcome of official medical diagnosis; and finally information about symptom type and duration since onset. Names can be anonymized.

In many embodiments, a MMHealth pipeline can be used for infectious disease diagnosis. In many embodiments, the array of sensors present on the MMHealth device can be used for a variety of different applications, including mental illness diagnosis. For mental health diagnosis applications, the pipeline can be adjusted to extract a wider range of features that have been shown to be indicators of, or have some correlation with, mental disorders. These can include pupil dilation, gaze aversion, emotion sentiment analysis, blinking rate and smile intensity and duration. In certain embodiments, the MMHealth pipeline can be applied for sports medicine in the monitoring of organs such as the lungs, and/or the health of joints. Fatigue can be a prominent feature set used in these applications, along with vital signs.

In many embodiments, the MMHealth pipeline can be used for the diagnosis of sleep disorders, including common ones such as sleep apnea. An MMHealth pipeline can be used for sleep studies that include monitoring of many different channels (including different vital signs) using wireless non-contact monitoring technology (rather than existing wired techniques) to better diagnose sleep disorders in the pediatric and geriatric populations. In many embodiments, a multimodal sensor stack can be used to expand remote diagnostic capacities for sleep disorders. In many embodiments, the MMHealth pipeline can include different features, including respiratory effort and the detection of limb movements, and in particular, limb movements in the legs.

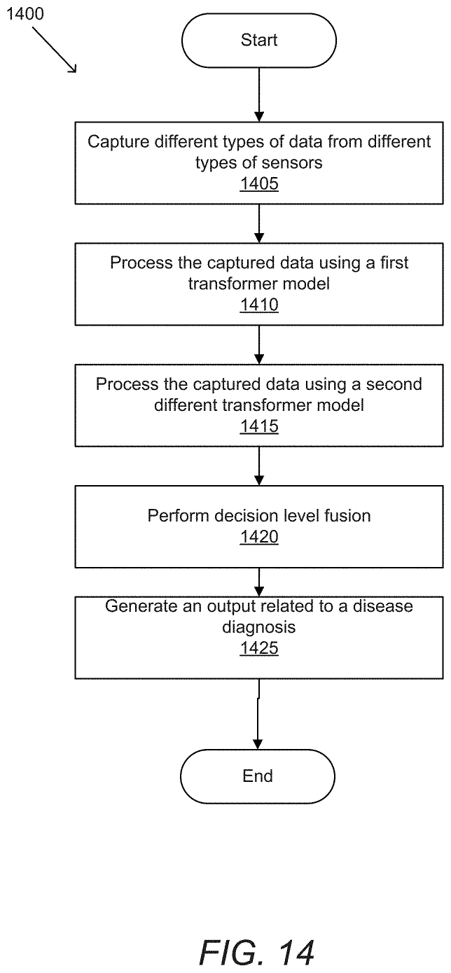

A process for disease diagnosis using a multi-modal pipeline in accordance with an embodiment of the invention is illustrated in FIG. 14. Process 1400 captures 1405 different types of data using different types of sensors. The different types of sensors can include an RGB camera that captures image data, a near infrared imaging (NIR) camera that captures NIR image data, a thermal imaging (TI) camera that captures thermal image data, an RF sensor that captures RF data, and an audio sensor that captures audio data.

The process 1400 generates 1405 a first vital sign estimate using a first process that includes using a multi-modal transformer model, wherein the multimodal transformer is used to train on the first type of data and the second type of data. In many embodiments, the process generates a final r-PPG signal by: generating a first r-PPG signal estimate using a first process that includes using a multi-modal transformer model, where the multimodal transformer is used to train on the image data, the NIR image data, and the thermal image data to obtain the r-PPG estimate. The process can generate a second r-PPG signal estimate using a second different process that includes estimating the second r-PPPG from RGB data, NIR data, and TI data separately and aggregating them together. The process then determines an r-PPG signal estimate based on the first r-PPG estimate and the second r-PPG signal estimate.

The process can generate 1410 a second vital sign estimate using a second different process that includes estimating the second vital sign estimate using the first type of data and the second type of data separately and then aggregating them together. The process can perform a respiratory rate process that generates a respiratory waveform signal by: generating a first respiratory waveform estimate using a multimodal transformer, where the multimodal transformer is trained on the NIR image data and the TI image data; generate a second respiratory waveform estimate from the NIR image data and the TI image data separately and then aggregating them together, and determine the respiratory waveform signal based on the first respiratory waveform estimate and the second respiratory waveform estimate.

The process can perform 1420 decision level fusion. In many embodiments, decision level fusion can using competing aggregator multimodal transformer models including a first model and a second model, wherein the first model receives as inputs the outputs from the r-PPG, the respiratory rate, and the blood oxygenation, and the second model receives as inputs raw data from the different types of sensors.

The process 1425 can generate 1425 an output related to a disease diagnosis. The process ends.

While specific processes for using multimodal transformers to diagnose disease described above, any of a variety of processes can be utilized to diagnose disease as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

Camera-Based Heart Rate Estimation from Facial Videos for Diverse Subject Skin Tones and Scenes

As noted above, heart rate (HR) is an essential clinical measure for the assessment of cardiorespiratory instability. In particular, smartphones that use in-built camera pipelines to measure HR from facial videos offer an economical solution in comparison to mass deployment of wearable sensors. However, existing computer vision methods that estimate HR from facial videos exhibit biased performance against dark skin tones. Accordingly, many embodiments provide for a physics-driven process that boosts performance on different skin tones. Furthermore, many embodiments can reduce errors due to lighting changes, shadows, and specular highlights and impart unbiased performance gains across skin tones, providing for non-contact HR sensing for patients across skin tones using smartphone cameras.

Contactless methods of HR estimation have been used, in which computer vision algorithms and artificial intelligence (AI) tools are used to remotely extract a blood volume pulse (BVP) signal and corresponding HR estimate from facial videos. Remote photoplethysmography (r-PPG) is a technique for HR estimation, however, the performance of certain r-PPG algorithms can fluctuate with changes in illumination condition, subject motion, and skin tone.

Accordingly, many embodiments provide for an r-PPG process that mitigates bias for skin tone. In particular, many embodiments account for the bias by considering imaging noise, and thus apply r-PPG denoising techniques to alleviate performance losses.

Many embodiments provide for an r-PPG process to estimate subject HR in a contactless manner using a smartphone camera. In many embodiments, the R-PPG can operate by looking for subtle color variations visible on the surface human skin, caused by sub-dermal light absorption fluctuations from changes in blood volume and content. Several known r-PPG algorithms have been used to extract the BVP signal from videos, including blind source separation (BSS), model-based, unsupervised data-driven, and supervised deep learning methods. However, these algorithms exhibit a performance gap, and therefore a bias, for certain types of skin tones, subject motions (e.g. speaking), and illumination conditions. Accordingly, many embodiments of the system address these biases as is useful for deployment of R-PPG technology in telemedicine applications.

Many embodiments of the system can address the biases in skin-tone, illumination conditions, and subject motions using physics-rooted techniques and camera noise analysis.

Many embodiments may use a Distance-PPG in which a weighted average of BVP signals from various facial regions-of-interest (ROI) are used with weighting in RGB space and by including a skin diffuse component weighting to address bias. This can enable a process in accordance with many embodiments to mitigate performance losses for subjects with darker skin tones, subjects in varying illumination conditions, and subjects who may be moving their face such as when they are talking.

The r-PPG Pipeline

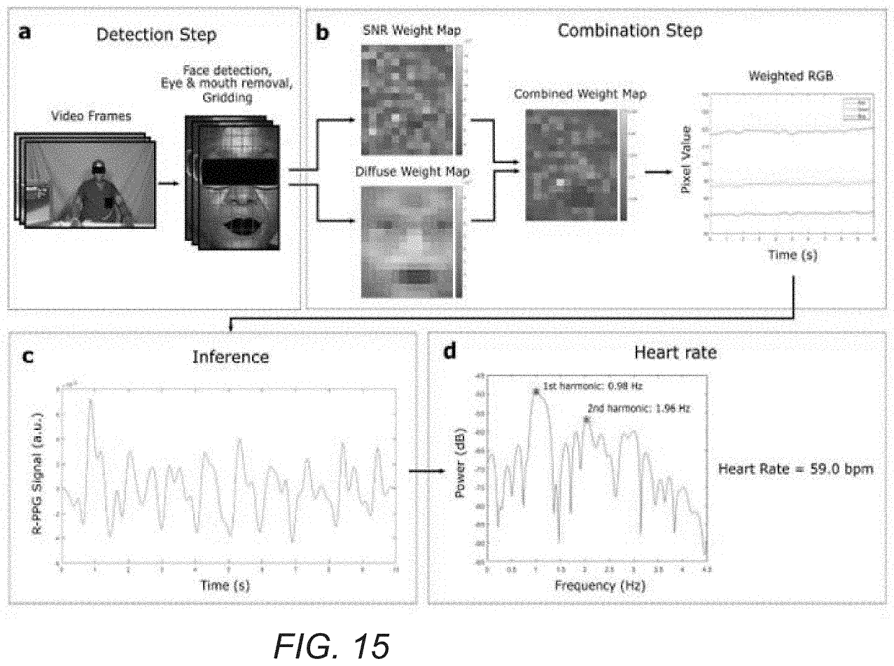

In many embodiments, an r-PPG pipeline can be used for HR estimation. There can be four components to a typical r-PPG pipeline: (a) detection, which identifies facial regions of interest in the video frame, (b) combination, which condenses the information from regions of interest into an RGB time series signal, (c) signal inference, which uses the time series signal to estimate the pulse volume waveform, and (d) HR estimation, which estimates the HR from the pulse volume signal. FIG. 15 illustrates a heart rate estimation process that includes the four components of a r-PPG pipeline where the combination step of the pipeline incorporates skin diffuse information weighting, in addition to SNR weighting in RGB space to achieve robust r-PPG performance across skin tones in accordance with an embodiment of the invention.

In many embodiments, a video can be passed through a neural network-based face detector, in order to identify the face region in the frame. Using feature point detectors, the eye and mouth regions can be identified and explicitly removed from the videos (since these regions do not contribute to the pulsatile signal), as illustrated as the detection step in FIG. 15. The next steps, namely combination, inference and HR step, can be carried out for smaller video-windows of a certain length (e.g., 10 seconds length with an overlap of 5 seconds).

For each video frame, the skin pixels can be combined to get one RGB sample for that time instance. Across all frames, after this combination, a time series RGB signal can be obtained, as illustrated in the combination step illustrated in FIG. 15.

The RGB signals can be then put through a signal inference technique. Many embodiments can use the CHROM algorithm. The output obtained from this step results in a pulsatile waveform estimate for each window, as illustrated as the inference step in FIG. 15.

The obtained pulsatile waveform can then be processed to arrive at the final HR, illustrated as the heart rate step in FIG. 15. Many embodiments can first filter the waveform using a Butterworth bandpass filter with pass band frequencies of, for example [0.7, 3.5] Hz. The power spectral density (PSD) can then be computed. Many embodiments can remove any temporal frequency artifacts that may be observed in an original video as a result of aggressive compression, likely due to unchanging green background. In many embodiments, a number (e.g., five) of the highest peaks in the PSD can be chosen. The peak with the highest combined fundamental and second harmonic power can be chosen as the one corresponding to the HR. The final HR for the video can be estimated as the average of the HR estimates for each (e.g., 10 second) window.

As noted above, many embodiments of the system for HR estimation provide for (i) weighting in RGB space, rather than blood volume signal space and (ii) skin diffuse component weighting.

RGB-space weighting: Existing spatial averaging methods estimate weights for each grid region, based on the blood volume signal quality. Instead of using these estimated weights to average the blood volume signals, as done in known methods, many embodiments can use these weights to average in RGB space. As a result, many embodiments can obtain one consolidated SNR weighted RGB signal, which can be again passed through the inference step to obtain the final blood volume signal.

(ii) Skin diffuse component weighting: In many embodiments, an image can be split into two constituent components: the diffuse component, that arises out of transmission and reflection through the skin, and a specular component, that arises from mirror-like surface reflections. Since the diffuse component contains the signal of interest, many embodiments utilize gridded diffuse components as additional weights. In particular, for each frame, the diffuse component can be estimated. It can then be gridded and averaged across the grid dimensions and time, in order to arrive at weights for each grid element.

The diffuse weights can play two roles in improving bias in performance as well as overall performance: first, it can remove specular affected regions from the average explicitly. Second, it can combat the sparsity issue observed in traditional SNR weights, since the diffuse component is continuous and non-sparse. The SNR weights and the diffuse weights can then be multiplied together and renormalized to arrive at the final spatial weights for the gridded video.

### Effect of Skin Tone on PPG Signal

In order to develop remote photoplethysmography (r-PPG) denoising and debiasing techniques, many embodiments derive potential sources of bias and links to statistical noise. Many embodiments may use a mathematical model for skin coloration, as a function of melanin content and blood volume fraction and extend this coloration model for the goal of analyzing response of the PPG signal to noise ratio (SNR), in the context of skin tone variation.

Let E(λ) represent the spectral power distribution of the light source concerned. Let S_{c}(λ) be the spectral sensitivity of a camera in use for color channel c. In many embodiments, the model can assume that light from the skin, as seen by the camera, emerges after two transmissions from the epidermis and one reflection from the dermis. That is, R=Tₑₚ²(λ)·R_{d}(λ). Using the expressions for T(λ) and R_{d}(λ), many embodiments can evaluate the value of R(λ), as a parametric function of fₘₑₗ (skin melanin fraction), f_{blood} (fraction of blood in the specific skin region) and f_{hg} (fraction of hemoglobin in the blood at the location). Then, the intensity captured in channel c by the camera can be given by f_{λ} E(λ)S_{c}(λ)R(λ)dλ.

Subsequently, R(λ) as R(λ, fₘₑₜ, f_{blood}, f_{hg}) can be referred to in order to incorporate all the relevant parameters. To understand the SNR as a function of radiance wavelength, many embodiments identify that the PPG signal arises out of temporal variation in the value of f_{blood}. The noise involved can be the noise involved in the capture process through the camera. First, many embodiments can look at the signal strength, while ignoring the effect of imaging noise. The PPG signal strength may be given by

L(λ)=|R(λ,fₘₑₜ,f_{blood}^{max},f_{hg})−R(λ,fₘₑₜ,f_{blood}^{min},f_{hg})|

That is, the strength of the PPG signal can be directly related to the radiance change that occurs between the maximum and minimum blood volume fraction in the face. Then, an estimate for the average signal strength for color channel c is given by

M_{c}(fₘₑₗ,f_{hg})=f_{λ}E(λ)S_{c}(λ)L(λ)dλ.

In analyzing the effect of skin tone, many embodiments can hold f_{hg} constant and evaluate the above signal strength metric for various reasonable values of fₘₑₗ. The values of all relevant physiological constants can be taken to be the average healthy values.

While the above descriptions and associated figures have depicted systems and methods for extracting vital signs using smartphone cameras and systems for multimodal health sensors any of a variety of configurations for extracting vital signs from smartphone cameras and systems for multimodal health sensors can be implemented in accordance with embodiments of the invention. More generally, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A multi-modal system for diagnosing disease, comprising:
a plurality of different types of sensors, including:
an RGB camera that captures image data;
a near infrared imaging (NIR) camera that captures NIR image data;
a thermal imaging (TI) camera that captures thermal image data;
at least one processor;
memory storing a disease diagnosis application, wherein the disease diagnosis application directs the processor to:
perform an r-PPG process that generates a final r-PPG signal by:
generating a first r-PPG signal estimate using a first process that includes using a multi-modal transformer model, wherein the multimodal transformer is used to train on the image data, the NIR image data, and the thermal image data to obtain the r-PPG estimate,
generating a second r-PPG signal estimate using a second different process that includes estimating the second r-PPPG from RGB data, NIR data, and TI data separately and aggregating them together,
determining the final r-PPG signal estimate based on the first r-PPG estimate and the second r-PPG signal estimate;
perform a respiratory rate process that generates a final respiratory waveform signal by:
generating a first respiratory waveform estimate using a multimodal transformer, wherein the multimodal transformer is trained on the NIR image data and the TI image data;
generating a second respiratory waveform estimate from the NIR image data and the TI image data separately and then aggregating them together; and
determine the final respiratory waveform signal based on the first respiratory waveform estimate and the second respiratory waveform estimate;
perform a blood oxygenation process that generates an oxygen saturation estimation;
perform a decision level fusion process that includes a plurality of competing aggregator multimodal transformer models including a first model and a second model, wherein the first model receives as inputs the outputs from the r-PPG process, the respiratory rate process, and the blood oxygenation process;
wherein the second model receives as inputs raw data from the plurality of different types of sensors.

2. The system of claim 1, wherein the blood oxygenation pipeline computes a band limited amplified skin reflectance variations at a 3D region of interest and adds to original grey value variations.

3. The system of claim 1, further comprising an
an RF sensor that captures RF data; and
an audio sensor that captures audio data;
wherein the disease diagnosis application direct the processor to perform an acoustic features process that:
divides audio data captured by the audio sensor into a first section with continuous speech and a second section with forced coughs;
train a first audio model using the first section; and
train a second audio model using the second section.

4. The system of claim 3, wherein a Poisson mask is applied to the audio data, wherein the Poisson mask equation is:
$$M ( {I}_{x} ) = P o i s s ( λ ) {I}_{x} \qquad ( l )$$
$$Poiss ( X = k ) = \frac{{λ}^{k} {exp}^{- k}}{k !}$$
wherein the Poisson Mask applied to a specific MFCC value Ix can be calculated by multiplying this value by a random Poisson distribution of parameters Ix and λ, where λ is the average value of the entire MFCC set.

5. The system of claim 1, further comprising a temperature pipeline.

6. The system of claim 1, wherein the decision level fusion process applies fuzzy aggregation to fuze different types of data and wherein a discrete Choquet Integral (CI) is used to fuze the classifier inputs and the highest confident class is selected, wherein the discrete Choquet Integral is:
$${C}_{g}^{j} ( d ) = {\sum }_{t = 1}^{T} {d}_{( ( t ) , j )} ( x ) [ g ( {A}_{t} ) - g ( {A}_{t - 1} ) ] ,$$
where C_{g}^{j}(d) is the integral for class j and fuzzy measure g, the inputs are sorted in decreasing order, and Aₜ is the set of inputs from (1) to (t).

7. The system of claim 1, wherein the decision level fusion process applies a Linear Order Static Neuron (LOSN) process.

8. A method for measuring vital signs, the method comprising:
identifying regions of interest (ROIs) from video data of an individual;
generating temporal waveforms from the ROIs;
analyzing the generated temporal waveforms to extract vital sign measurements; and
generating outputs based on the analyzed temporal waveforms.

9. The method of claim 8 further comprising capturing the video, wherein capturing the video comprises:
capturing video of an individual;
analyzing the captured video to determine whether a quality exceeds a given threshold; and
when the quality does not exceed the given threshold, providing instructions to recapture the video.

10. The method of claim 8, further comprising processing the captured video, wherein processing the captured video comprises at least one of illumination normalization and motion stabilization.

11. The method of claim 8 further comprising generating a motion cue video and a color cue video, wherein processing the color cue video comprises motion stabilization and processing the motion cue video does not include motion stabilization.

12. The method of claim 8, wherein identifying ROIs from video data comprises performing segmentation using a convolutional neural network (CNN).

13. The method of claim 8, wherein generating temporal waveforms from the ROIs comprises:
tracking motion of facial features within the ROIs between frames of video; and
calculating velocity vectors based on the tracked motion.

14. The method of claim 8, wherein analyzing the generated temporal waveforms to extract vital sign measurements comprises performing component analysis and frequency filtering to identify patterns in the video.

15. The method of claim 14, wherein the identified patterns comprise at least one of changes in at least one channel of the video data and periodic motion in the head and neck region.

16. The method of claim 8, wherein generating outputs based on the analyzed temporal waveforms comprises providing a notification when at least one of the vital sign measurements exceeds a given threshold.

17. A multi-modal system for diagnosing disease, comprising:
a plurality of different types of sensors that capture different types of data, including a first type of data and a second type of data;
generate a first vital sign estimate using a first process that includes using a multi-modal transformer model, wherein the multimodal transformer is used to train on the first type of data and the second type of data;
generate a second vital sign estimate using a second different process that includes estimating the second vital sign estimate using the first type of data and the second type of data separately and then aggregating them together;
perform decision level fusion;
generate a disease diagnosis.

18. The multi-modal system of claim 17, wherein the first process comprises generating a first r-PPG signal estimate that includes using a multi-modal transformer model, wherein the multimodal transformer is used to train on image data, NIR image data, and thermal image data to obtain the first r-PPG estimate;
wherein the second process comprises generating a second r-PPG signal estimate that includes estimating a second r-PPPG from RGB data, NIR data, and TI data separately and aggregating them together.

19. The multi-modal system of claim 18, further comprising performing a respiratory rate process that generates a final respiratory waveform signal by:
generating a first respiratory waveform estimate using a multimodal transformer, wherein the multimodal transformer is trained on NIR image data and the TI image data;
generating a second respiratory waveform estimate from the NIR image data and the TI image data separately and then aggregating them together; and
determine the final respiratory waveform signal based on the first respiratory waveform estimate and the second respiratory waveform estimate.

20. The multi-modal system of claim 19, further comprising performing a blood oxygenation process that generates an oxygen saturation estimation;
perform a decision level fusion process that includes a plurality of competing aggregator multimodal transformer models including a first model and a second model, wherein the first model receives as inputs the outputs from the r-PPG process, the respiratory rate process, and the blood oxygenation process;
wherein the second model receives as inputs raw data from the plurality of different types of sensors.
